(19) United States
(12) Patent Application Publication
(10) Pub. No.: US 2025/0322695 A1
(43) Pub. Date: Oct. 16, 2025

(54) DETECTING AND ANALYZING STUDENT LEARNING PATTERNS

(71) Applicant: 2hr Learning, Inc., Austin, TX (US)

(72) Inventors: Naman Yadav, Jaipur (IN); Dorukhan Tokay, Istanbul (TR); Harsh Arya, Jaipur (IN); Pedro Ricardo Gomes Dias, Hong Kong (CN)

(73) Assignee: 2hr Learning, Inc., Austin, TX (US)

(21) Appl. No.: 19/177,496

(22) Filed: Apr. 11, 2025

Related U.S. Application Data
(60) Provisional application No. 63/633,017, filed on Apr. 11, 2024.

(51) Int. Cl. G06V 40/20; A61B 5/16; G06T 7/70; G06V 10/764; G06V 10/98; G06V 40/16

(52) U.S. Cl. CPC G06V 40/20; G06T 7/70; G06V 10/764; G06V 10/993; A61B 5/163; A61B 5/168; G06T 2207/30201; G06V 40/174

(57) ABSTRACT

A user learning pattern detection system and method to guide an Artificial Intelligence (AI) engine to identify and analyze user learning behaviors, specifically anti-patterns (negative patterns) and posi-patterns (positive patterns), within an online learning platform is disclosed. The user learning pattern detection method involves collecting diverse data, including media streams (e.g., webcam feed, microphone audio), user interaction (e.g., keystrokes, mouse clicks), and engagement metrics. This data is then analyzed to generate insights into the user's learning behavior. Using these insights, prompts are generated and provided to the AI engine, which employs machine learning algorithms and computer vision techniques to detect and classify learning behaviors. The detected patterns undergo a quality check using multimodal large language models (LLMs) to ensure accuracy. Finally, the method generates detailed reports, including video clips of key moments, verifying the patterns, and offering user recommendations to enhance the learning experience.

### CROSS-REFERENCE TO RELATED APPLICATION

This application claims the benefit under 35 U.S.C. § 119 (e) and 37 C.F.R. § 1.78 of U.S. Provisional Application No. 63/633,017, filed Apr. 11, 2024, which is incorporated by reference in its entirety.

### FIELD OF THE INVENTION

The present invention generally relates to the field of electronics, and more specifically to a system of detecting and analyzing the learning patterns of a user, which includes anti-patterns or posi-patterns, when the user is using any online learning platforms.

### BACKGROUND OF THE INVENTION

Traditionally, quality control of antipattern clips in online learning environments was a labor-intensive process requiring human observers to manually review and analyze video footage. These human reviewers had to meticulously watch each clip to identify errors or inconsistencies, such as moments when a student might display signs of disengagement or confusion. This manual approach was not only time-consuming, often taking hours or even days to process large volumes of data, but it was also prone to human error. Factors like reviewer fatigue, subjective judgment, and the sheer volume of content could lead to mistakes or inconsistencies in the analysis, resulting in inaccurate assessments of student behavior.

In learning behavior analysis, the reliance on human reviewers meant that any findings had to be manually checked and verified, a process that was slow and could introduce further errors. Human reviewers might miss some patterns or misinterpret certain behaviors due to the limitations of manual observation. Moreover, the results of these analyses could vary depending on the individual reviewer's experience, leading to inconsistencies in the quality control process.

Earlier systems used in this context typically focused on a single data source, such as analyzing test performance without considering other important aspects like how a student interacted with the learning material during the session. For instance, a system might evaluate a student's test scores without accounting for behavioral data like how often the student paused the video, how much time they spent on each section, or their level of engagement during the lesson. This approach often resulted in an incomplete picture of the student's learning experience, as it overlooked the broader context of their behavior.

Additionally, such systems were generally not designed specifically for educational platforms, which meant they could not fully capture the complexities of student learning behaviors. The absence of integrated data sources, such as combining screen monitoring, time analytics, and engagement metrics with traditional performance data, limited the effectiveness of these systems in providing accurate insights.

### SUMMARY

One or more embodiments of a method include:
executing code using one or more processors of a computer system to cause the computer system to perform operations comprising:
   collecting media stream data, user interaction data, and user engagement data, wherein the media stream data includes webcam feed, microphone audio, screen captures, and system audio, and user interaction data includes keystrokes, mouse clicks, URLs visited, active application data, active window data, and window titles;
   analyzing the pre-processed data to generate insights that indicate the learning behavior of the user using the online learning platform;
   guiding and constraining an AI engine to perform operations comprising:
      detecting the learning behaviors of the user using the online learning platform using machine learning algorithms and computer vision techniques;
      classifying the detected user's learning behavior into positive learning patterns (posi-patterns) and negative learning patterns (anti-patterns);
      performing a quality check on the detected anti-patterns and posi-patterns using multimodal large language models (LLMs), to verify the accuracy and relevance of the detected patterns;
   generating reports that verify anti-patterns and posi-patterns and provide recommendations to the user, wherein the reports include a video clip featuring the section where anti-pattern or posi-patterns occurred.

One or more embodiments of a system include:
one or more processors;
a memory, coupled to the one or more processors, storing code that when executed by the one or more processors cause a computer system to perform operations comprising:
   collecting media stream data, user interaction data, and user engagement data using a data collector, wherein the media stream data includes webcam feed, microphone audio, screen captures, and system audio, and user interaction data includes keystrokes, mouse clicks, URLs visited, active application data, active window data, and window titles;
   analyzing the collected data using an analyzer to generate insights that indicate the learning behavior of the user using the online learning platform;
guiding and constraining an AI engine to perform operations comprising:
detecting the learning behaviors of the user using the online learning platform using a learning pattern detector that utilizes machine learning algorithms and computer vision techniques;
classifying the detected user's learning behavior into positive learning patterns (posi-patterns) and negative learning patterns (anti-patterns) using a classifier;
performing a quality check using a quality checker on the detected anti-patterns and posi-patterns using multimodal large language models (LLMs), to verify the accuracy and relevance of the detected patterns;
generating reports that verify anti-patterns and posi-patterns and provide recommendations to the user, wherein the reports include a video clip featuring the section where anti-pattern or posi-patterns occurred.

### BRIEF DESCRIPTION OF THE DRAWINGS

The systems and methods described herein may be better understood, and their numerous objects, features, and advantages are made apparent to those skilled in the art by referencing exemplary embodiments depicted in the accompanying figures. The use of the same reference number throughout the several figures designates a like or similar element.

FIG. 1 depicts an exemplary user learning pattern detection system based on media stream data and user data analytics.

FIG. 2 depicts an exemplary user learning pattern detection process based on media stream data and user data analytics.

FIG. 3 depicts an exemplary report and video clips featuring the user learning patterns generation process, which is an embodiment of the user learning pattern detection process based on media stream data and user data analytics of FIG. 2.

FIG. 4 depicts an exemplary anti-patterns quality check process, which is an embodiment of the user learning pattern detection process based on media stream data and user data analytics of FIG. 2.

FIG. 5 depicts the common user learning patterns that are taken into consideration by the user learning pattern detection system based on media stream data and user data analytics.

FIG. 6 depicts an exemplary network environment in which the system of FIG. 1 and the process of FIG. 2 may be practiced.

FIG. 7 depicts an exemplary computer system.

### DETAILED DESCRIPTION

A user learning pattern detection system to guide an Artificial Intelligence (AI) engine to detect the anti-patterns (negative patterns), and posi-patterns (positive patterns) from the user's learning behavior during an online learning session is disclosed. The user learning pattern detection system includes an online learning platform using which the user access the online learning sessions, and a learning pattern analysis module. The online learning platform and the learning pattern analysis module are operatively coupled to each other. The learning pattern detector includes a data collector integrated within it which collects the user interaction data, user engagement data, and media stream data. The collected data is then analyzed using an analyzer, which is configured to generate the insights that help a prompt generator to generate the prompts.

The prompt generator utilizes the analyzed data to populate the prompt structure provided by the prompt engineer for prompt generation. These prompts are then used by the AI engine to detect the anti-pattern and posi-patterns. The AI engine has a learning pattern detector integrated within it to detect the user's learning behavior, which includes identifying the anti-patterns and posi-patterns in the user's behavior. The detected learning patterns are then classified into anti-patterns and posi-patterns using a classifier.

The classified anti-patterns and posi-patterns are then passed through a quality check using a quality checker, which is configured to check whether the detected anti-pattern or posi-pattern is correct or not, or if there are any errors or any discrepancies during the detection process. Upon the proper quality check of the learning behavior of the user, the AI engine generates a report that includes video clips, reports, and recommendations. The video clip features the video at which the anti-pattern or posi-pattern occurred. The user can access this video clip using a hyperlink provided to the user in the report.

The user learning pattern detection system offers several advantages, including the ability to accurately detect and classify user learning behaviors into positive (posi-patterns) and negative (anti-patterns) patterns using advanced AI techniques. By integrating multimodal data such as media streams, user interactions, and engagement metrics the user learning pattern detection system provides a comprehensive analysis of the user's learning experience. This enables personalized feedback, allowing users to receive targeted recommendations that enhance their learning efficiency. The inclusion of quality checks ensures the accuracy and relevance of detected patterns, minimizing errors and improving the reliability of the insights provided. Additionally, the generated reports, complete with video clips, offer a clear and actionable understanding of user behaviors, ultimately leading to more effective and are generated in correspondence to the user's online learning experiences.

The system and method set forth herein address technical issues with generating the desired outputs described herein. Conventionally, manual processes were used to generate the desired outputs and were very tedious and time consuming. The present system and method utilize an automated system that does not merely automate a manual process or use a conventional system in a conventional way. The present system and method utilize one or more artificial intelligence (AI) engines and integrate programmatic process management to technologically guide and constrain the one or more AI engines to produce the desired outputs in a completely different way than both any manual process and different than normal use of programs and AI engines. Utilizing specially engineered guidance and control to direct an AI system to solve the problems below presents a technical problem that requires a technical solution. The system and method described below are not simply engaging a computer to carry out conventional mental processes, but rather change how computers (and AI systems, specifically) operate to achieve the generation results that were not previously possible or were substantially inefficient prior to the system and method set forth below. The AI system needs specific technical guidance, control, and constraints to achieve results that are not otherwise achievable.

Prompts are used to guide and constrain each AI engine. The prompts guide each AI engine by steering the AI engine(s). “Guiding” an AI engine refers to providing the AI engine with a general direction or framework to shape the AI engine's behavior or decision-making process. Guiding sets goals or principles. Guiding allows the AI engine some flexibility to interpret and adapt, much like giving it a compass to navigate rather than a fixed path.

Constraining each AI engine includes imposing specific, hard limits or rules on what each AI engine can do. Constraining an AI engine can also include providing specific input data to not only guide but also constrain the scope of each AI engine's reasoning basis and response. Constraining each AI engine assists with aligning the AI engine(s) for its (their) intended use.

Normally AI engines are provided a single user prompt requesting the AI engine, such as OpenAI's ChatGPT and its various implementations such as Anthropic's Claude Sonnet, to perform a task and produce an output. However, this conventional AI engine prompting method has a variety of technical shortcomings. Without proper guidance and constraints, an AI engine will not produce the desired output specified as produced by the system and method described herein. Instead, the AI engine will produce many unusable outputs that are unusable for a variety of reasons including so-called “hallucinations” where the AI engine presents fabricated information, duplicate outputs, too few outputs, too many outputs, outputs that do not meet desired criteria, and so on. Without special technical guidance, the AI engine cannot reliably be applied to generate desired outcomes.

The system and method generate decomposed, technically engineered AI prompts to include selected and integral AI engine guidance and constraints. The technically engineered prompts are generated and guided with programmatic, automatic inputs specifically designed to unconventionally guide and constrain an AI engine to produce desired outputs, perform quality control to retain or automatically discard outputs that do not meet guidance and constraints, and make the desired outputs available for use, such as use by computer system applications. In at least one embodiment, the problem to be solved by the integrated programmatic and AI engine system and method is uniquely and unconventionally decomposed, and AI prompts are used to solve the decomposed problem. Furthermore, the programmatic inputs to the decomposed AI prompts provide guidance to meet desired output characteristics.

Determining a number of prompts, the guidance and constraints within each prompt, and data flowing from one AI engine prompt to another, in addition to testing a number of prompts for the decomposed problem, testing within each prompt, and validating a desired quality of outputs becomes an intractable combinatorial problem without technical guidance and constraint of the system and method described herein. Thus, the present system and method described implement an integration of programmatic management over decomposed prompts with engineered AI engine guidance and constraints to effect an improvement in AI, programmatic AI management, and AI integrated with programmatic management technology. The present system and method allow computer systems to include programmatic management, one or more AI engines, and one or more data sources to produce the output described herein that previously could not be produced with conventionally prompted AI engines or could only be produced by humans utilizing a completely different, time consuming, and tedious process. The system and method improve conventional methods through the use of a programmatic AI engine management system to generate decomposed, technically engineered AI prompts to include selected and integral AI engine guidance and constraints. It is, for example, the incorporation of the programmatic AI engine management system to generate decomposed, technically engineered AI prompts to include generated, integral, and unconventional AI engine guidance and constraints and execution by the one or more AI engines to provide useful results that improve existing technical processes, which is not an automation of a conventional process.

Programmatic components and AI engines generally utilize one or more processors that have access to memory, which may include one or more storage components, to execute and perform functions. An AI engine is a core hardware and software system that enables artificial intelligence applications to process data, learn patterns, and generate insights or actions. It functions as the brain behind AI-driven systems, facilitating tasks such as machine learning, natural language processing, and decision-making. Exemplary components of an AI engine are:
1. Machine Learning Models-Algorithms that analyze data, recognize patterns, and make predictions.
2. Neural Networks-Deep learning architectures that mimic the human brain for tasks like image and speech recognition.
3. Data Processing Module-Handles raw data input, transformation, and feature extraction.
4. Inference Engine-Applies trained models to make real-time decisions based on new data.
5. Optimization Algorithms-Improves model efficiency, reducing errors and improving predictions.
6. Natural Language Processing (NLP) Module-Enables AI engines to understand, interpret, and generate human language (e.g., chatbots, voice assistants).
7. Computer Vision Module-Allows AI to interpret and analyze images or videos.
8. Reinforcement Learning Mechanism-Helps AI learn from trial and error, optimizing performance over time.
9. API Interface-Connects the AI engine with applications, enabling integration with other software or platforms.

Examples of AI Engines include: XAI's Grok and variations thereof, Google TensorFlow, Meta's PyTorch, Microsoft Azure AI, OpenAI's ChatGPT and variations thereof, IBM Watson, OpenAI Whisper, Google BERT & T5, Amazon Lex, Anthropic Claude, DeepMind's AlphaCode, Google Vision AI, Meta's DINO & SAM (Segment Anything Model), NVIDIA DeepStream. OpenCV AI Kit, Amazon Polly. Google WaveNet, Deepgram.

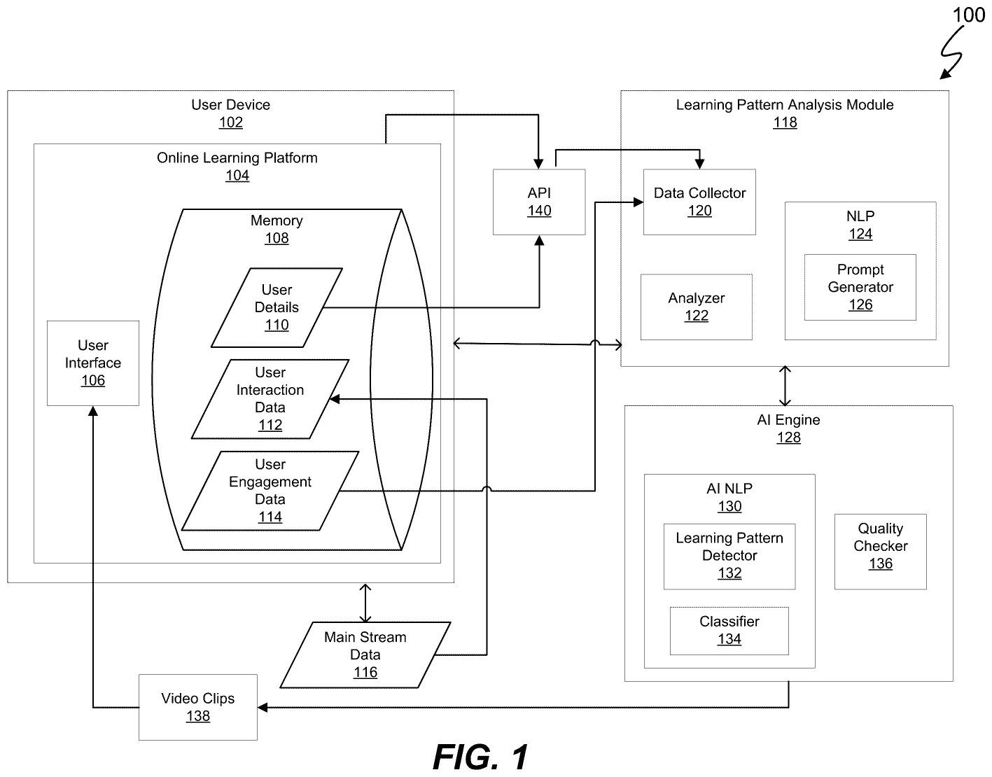

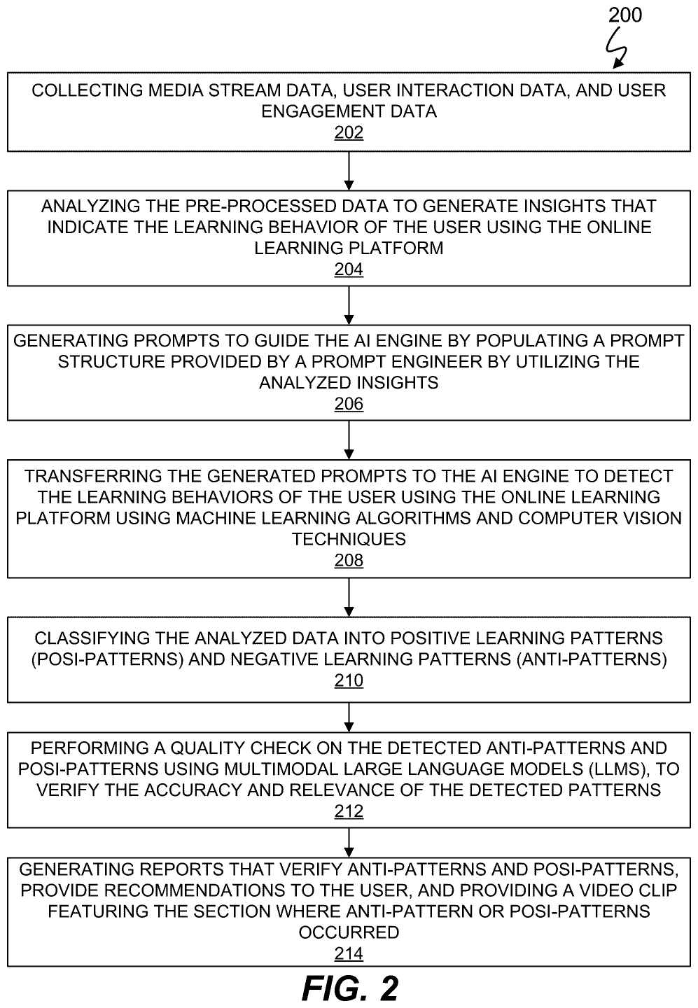

FIG. 1 depicts an exemplary user learning pattern detection system 100 based on media stream data 116 and user data analytics. FIG. 2 depicts an exemplary user learning pattern detection process 200 based on media stream data 116 and user data analytics, utilizing the user learning pattern detection system 100.

Referring to FIGS. 1 and 2, in operation 202, a data collector 120 collects user interaction data 112, user engagement data 114, and media stream data 116.

The data collector 120 is integrated within a learning pattern analysis module 118, further operatively coupled to an online learning platform 104. The user can access the online learning platform 104 via, a user device 102. The user device 102 may include a computer, smartphone, tablet, iPad, laptop, or any other compatible device to access the online learning platform 104.

The data collector 120 is designed to capture a broad range of data, including user interaction details 112, user engagement details 114, and media stream data 116. The data collector 120 collects media stream data 116 that include webcam video, microphone audio, screen captures, and system audio using devices like webcam, and microphone (not shown in the figure). This media stream data 116 is gathered from devices that might have integrated webcams and microphones or ones connected externally. The data collection ensures that both visual (webcam footage) and on-screen activities are recorded simultaneously, providing a comprehensive view of the user's environment and actions.

In addition to media stream data 116, the data collector captures detailed user interaction data 112, such as keystrokes, mouse clicks, URLs visited, and information about the applications and windows the user interacts with, including their titles by utilizing the data from the keyboard, and mouse. The keyboard and mouse are either integrated within the user device 102 or operatively coupled to the user's device 102. This helps in understanding how users interact during online learning sessions.

The user engagement data 114 includes browsing history, test scores, rates of assignment completion, and the amount of time users spend on specific tasks. The user engagement data 114 is vital for assessing how deeply and effectively users engage with the educational material during the online learning session. The data collector 120 also tracks the exact time and context in which questions are asked during online learning sessions and gathers detailed quiz information, such as the time taken to complete quizzes and the accuracy of the answers.

Furthermore, an API 140 is operatively coupled to the online learning platform 104 and the learning pattern analysis module 118. The API 140 is configured to provide access to a wide array of metrics. This API 140 delivers detailed lists of URLs visited and other user-specific details, ensuring that all aspects of user interaction and engagement can be analyzed in depth.

In operation 204, an analyzer 122 analyzes the collected data to generate insights that indicate the learning behavior of the user using the online learning platform 104.

The data collected by the data collector 120 is pre-processed before passing it to the analyzer 122 for further analysis. Pre-processing the collected data involves organizing and refining the raw data into a structured format that is ready for analysis. It includes data cleaning, where any inconsistencies, errors, or missing values in the raw data are identified and corrected. This might involve removing duplicate entries, filling in missing data points, or standardizing different data formats to ensure consistency across the dataset. This structured, pre-processed data enables more accurate, efficient, and insightful analysis, ultimately leading to better decision-making and outcomes.

The pre-processed data is then passed on to the analyzer 122 for further analysis. The analyzer 122 utilizes advanced computer vision techniques to analyze video recordings and detect patterns in the user's learning behavior. The analyzer 122 is integrated within the learning pattern analysis module 118. The analyzer 122 is further configured to utilize gaze detection technology to monitor the gaze of the user while using the online learning platform 104. The analyzer 122 monitors the direction of the user's gaze while they interact with content on the online learning platform 104. This gaze data is critical for assessing the level of visual engagement the user has with the educational material. The analyzer 122 analyzes whether the user is actively focusing on the content or if their attention is wandering away from the screen.

This detailed gaze analysis is then integrated into a broader assessment of the user's learning behavior. By combining gaze data with other behavioral indicators, the AI engine 128 can identify potential anti-patterns, such as signs of distraction or lack of focus, which may indicate disengagement. Conversely, it can also recognize posi-patterns, such as sustained attention and consistent engagement, which suggest that the user is effectively concentrating on the learning material.

The insights derived from the analysis of the collected data, including user interaction data 112, user engagement data 114, and media stream data 116, and the gaze data play a crucial role in generating prompts.

In operation 206, a prompt generator 126 guides the AI engine 128 by populating a prompt designed by a prompt engineer and by utilizing the analyzed insights. The prompt guides and constrains the AI engine 128 to transform input data includes the insights into an output. The prompt generator 126 fetches the analyzed data from the analyzer 122 and populates the prompt.

The prompt generator 126 utilizes NLP (Natural Language Processing) techniques by using a NLP 124 to generate the prompts that are provided to the AI engine 128. The prompt generator 126 utilizes the analyzed data from the analyzer 122, and the prompt structure provided by the prompt engineer, which includes the prompt structure, and rules and guidelines to create the prompt. The prompt generator 126 is integrated within the learning pattern analysis module 118 and is operatively coupled to a learning pattern detector 132, integrated within the AI engine 128. The prompt generator 126 utilizes the analyzed data and populates the prompt structure using that data.

In operation 208, the prompt generator 126 transfers the generated prompts to the AI engine to detect the learning behaviors of the user using the online learning platform 104. The AI engine 128 utilizes the learning pattern detector 132 which incorporates machine learning algorithms and computer vision techniques to detect the learning behaviors of the user.

The AI engine 128 utilizes a Vision Large Language Model (LLM-V), which is an AI model that can interpret and understand both images and text in combination. LLM-V is a multimodal large language model that can perform hundreds of vision-language tasks, such as visual perception, generation, and understanding. It was released in June 2024 on GitHub. LLM-V is a type of multimodal AI that combines semantic processing and machine vision to understand images. They can learn from both images and text simultaneously to perform tasks like image captioning and visual question answering. LLM-V is important because it helps bridge the gap between how humans think about the world and visual representations.

This capability allows the AI engine 128 to process and analyze multimodal data, which means it can seamlessly integrate and make sense of information that comes from different sources, such as visual data from images or videos and textual data from accompanying descriptions or context. By understanding these multiple forms of data together, the AI engine 128 can draw more accurate conclusions, enhancing its ability to interpret complex learning environments where both visual cues and textual information are essential.

The AI engine 128 has the learning pattern detector 132 within it, which utilizes advanced machine learning algorithms designed to automatically identify patterns of user behavior during learning sessions. These algorithms analyze data derived from video recordings, user interactions, and other relevant sources to detect anti-patterns-behaviors that indicate issues like distraction, lack of engagement, or improper learning habits. Simultaneously, the learning pattern detector 132 can also recognize posi-patterns, which are indicative of positive learning behaviors, such as sustained attention, active engagement, or effective interaction with the content.

By combining the use of LLM-V with these machine learning algorithms, the AI engine 128 not only processes and analyzes data more effectively but also provides deeper insights into the learning process.

In operation 210, a classifier 134 classifies the detected learning behavior of the user into positive learning patterns (posi-patterns) and negative learning patterns (anti-patterns).

The classifier 134 plays a crucial role in analyzing and categorizing the learning behavior of a user by examining the data collected during their interaction with educational content. The classifier 134 is integrated within the AI engine 128 and is operatively coupled to the learning pattern detector 132. The classifier 134 classifies this behavior into two main categories: positive learning patterns (posi-patterns) and negative learning patterns (anti-patterns).

Anti-patterns and posi-patterns are concepts used to describe behavioral patterns, particularly in the context of learning, where they represent negative and positive behaviors, respectively.

Anti-patterns refer to behaviors or practices that are counterproductive, inefficient, or detrimental to achieving desired outcomes. In the context of learning, antipatterns might include actions such as frequent distraction, lack of focus, skipping important content, or engaging in activities that do not contribute to learning goals. For instance, if the user often looks away from the screen during an online lecture or spends time on unrelated websites while a lesson is ongoing, these behaviors would be considered antipatterns. These patterns indicate a problem in the learning process that could hinder progress or understanding.

Posi-patterns, on the other hand, represent positive and effective behaviors that contribute to successful outcomes. In a learning environment, posi-patterns might include sustained attention to the content, active participation in discussions or quizzes, and consistently engaging with the material in a meaningful way. For example, if a student maintains eye contact with the screen during a lecture, takes notes, and spends adequate time on assignments, these behaviors would be classified as posi-patterns. These patterns reflect good learning habits that are likely to lead to better comprehension and retention of the material.

Identifying anti-patterns and posi-patterns is important for improving learning experiences. By recognizing these patterns, the AI engine 128 can provide feedback or interventions to correct negative behaviors and encourage positive ones, ultimately enhancing the overall effectiveness of the learning process.

For example, if a user consistently maintains focus on the screen, regularly participates in quizzes, and spends appropriate amounts of time on assignments, the classifier 134 might identify these as posi-patterns, indicating effective learning and engagement. Conversely, if the user frequently looks away from the screen, skips parts of the content, or shows signs of distraction such as rapid switching between windows or browsing unrelated websites, the classifier 134 would categorize these behaviors as anti-patterns.

The learning pattern detector 132 detects the timestamps where the change in the behavior of the user is observed. The classifier 134 utilizes this data and compares it with the pre-stored parameters which act as a measure of the anti-patterns and posi-patterns.

In operation 212, a quality checker 136 performs a quality check on the detected anti-patterns and posi-patterns using multimodal large language models (LLMs), to verify the accuracy and relevance of the detected patterns.

Quality checker 136 utilizes an advanced mechanism that performs a thorough quality assessment of detected anti-patterns and posi-patterns by utilizing multimodal large language models (LLMs). Multimodal large language models (MM-LLMs) are AI models that can process and understand multiple types of data, including text, images, audio, and video.

The primary purpose of the quality check is to ensure the accuracy and relevance of the identified patterns. The quality check begins by determining whether the current anti-pattern being analyzed is supported by the automated quality checker 136. If the anti-pattern is not supported, it automatically passes the QC, allowing it to move forward without further checks.

The first check involves detecting session drift, where the quality checker 136 examines the video by capturing frames at regular intervals from the start to the end of the session. These frames are then analyzed using tools like OpenAI's GPT model to verify whether the necessary question or explanation transitions occurred as expected. If the GPT model confirms these transitions, the video passes this check.

The second check focuses on detecting any external sources being used during the session, such as Google, ChatGPT, or other tools that might indicate cheating. The quality checker 136 analyzes each captured frame for signs of such external sources by analyzing the media stream data 116, which includes the details of the visited URLs. If any are detected, the video fails this check, ensuring that the session's integrity is maintained.

After all these checks are completed, the quality checker 136 evaluates the results. If the video passes all the checks, it is deemed to have passed the quality control, and the process returns a positive result. Conversely, if the video fails any of the checks, it is marked as failing the QC, ensuring that only high-quality, relevant patterns are considered accurate.

Further, the quality checker 136 also utilizes OCR techniques to assess the relevance of the video clips 138. OCR accuracy refers to the ability of optical character recognition software to produce machine-readable text content from scanned images or PDF files that exactly match the letters, numbers, symbols, and words in the original document. Optical Character Recognition (OCR) is a technology that not only extracts the text displayed on a screen but also provides the precise positions of this text within the image. The positions are often given as pixel coordinates, which define a bounding box around the text, usually specified by the top-left corner's coordinates along with the width and height, although other formats can be used as well. The OCR thus generates a list of objects where each object contains both the text and its corresponding location on the screen.

This step is repeated for each sampling time, i.e., at every specific interval, the OCR captures and records the text and its position on the screen. To track changes over time, the detected objects from the previous sampling frame are compared with those from the current frame. However, instead of directly comparing the text, a technique called fuzzy matching is used. Fuzzy matching is particularly useful because OCR can sometimes misinterpret characters like mistaking an ‘L’ for an ‘T’.

The quality check is done using multiple ways and the quality check also varies from application to application. For instance, in the case of the IXL application, which is a personalized learning program that helps students learn and practice math, English, science, social studies, and Spanish. It's available for grades Pre-K-12. The user has to scroll down the page within a predefined time and click on a guide button to go to the next page. When the user clicks on the guide button it is assumed by the quality checker 136 that the user has read the whole content with attention. However, if the user scrolls the page very fastly, i.e., the scroll time is less than the predefined threshold time of scrolling the page, then the quality checker 136 disapproves it and the video clips 138 shared by the learning pattern detector 132 are classified under the anti-pattern category.

Further, in the case of other applications like Khan Academy, the use of a guide button is not there. The user has to just scroll through the page within the predefined scrolling time.

The algorithm to perform the quality check on the detected anti-patterns and posi-patterns is given below:
1. Check if the current AntiPattern called is supported by automated QC
   1.1 If not supported, then return True (passes QC by default)
   1.2 If supported, proceed further
2. Check for Session Drift:
   2.1 Determine the start and end frames of the video
   2.2 Capture a frame every few seconds within this range
   2.3 Call the OpenAI's GPT model to analyze each captured frame
      The model checks if the required question or explanation transition happened in the sequence
   2.4 If the GPT model confirms a transition, the videos passes this check.
3. Check for Detection of External Sources (Cheating):
   3.1 For each captured frame, the GPT model checks if the user was using any external sources such as google, chatgpt, etc.
   3.2 If any such instance is found, the video fails this check.
4. Check if the IXL explanation was fully scrolled by the user:
   4.1 For each captured frame, the GPT model checks if the user has fully scrolled through the explanation
   4.2 If the user has not fully scrolled, then the video fails this check.
5. If the video passed all three checks, return True (Videos passes QC)
   If the video failed any check, return False (Video fails QC)

In operation 214, the AI engine 128 generates reports that verify anti-patterns and posi-patterns and provide recommendations to the user. The reports include a video clip 138 featuring the section where anti-pattern or posi-patterns occurred.

The generated reports include video clip 138 featuring only that part of the online learning session where the learning pattern detector 132 has detected anti-patterns or posi-patterns. The generated reports further include recommendations and a detailed report, which includes the advice given to the user if they are not focusing during the online learning session, and praising the user if the user is actively participating during the online learning session. The report includes details of the timestamp at which the learning pattern detector 132 has detected the anti-pattern or posi-pattern, along with this the report also includes a hyperlink using which the user can access the video clip 138 of the corresponding anti-pattern or posi-pattern. The recommendations address specific behaviors detected during the user's online learning session and suggest corrective actions to improve learning efficiency.

The generation of reports begins by selecting specific segments from video recordings that visually capture the identified patterns in a user's behavior. These patterns can be either anti-patterns, such as moments of user distraction, or posi-patterns, like instances of active participation. By carefully choosing clips that clearly illustrate these patterns, the AI engine 128 ensures that the most relevant timestamps are highlighted for further review.

Once the timestamps are selected, the next step involves generating video clips that specifically emphasize these anti-patterns and posi-patterns. These clips are designed to focus on the detected behaviors, providing a clear visual representation of the user's learning habits. For example, a clip might show a user frequently looking away from the screen, signaling distraction, or it might show a user actively engaging with the content, indicating strong participation. The generation of these clips is an essential step in transforming raw video data into actionable insights that can be easily interpreted.

After the relevant clips are generated, they are provided to educators along with detailed analysis reports. These reports explain the context of the observed patterns, offering educators a comprehensive understanding of the user's behaviors. The combination of visual evidence and analytical insights allows educators to review the specific moments where the user either struggled or excelled. This information can then be used to tailor teaching strategies, addressing areas where users are prone to distraction or reinforcing behaviors that indicate active engagement. By having access to both the video evidence and contextual analysis, educators are better equipped to make informed decisions that can enhance the learning experience and improve educational outcomes.

The output reports, including the video clips 138, reports, and recommendations are provided to the user in the JSON format. The output is stored in the cloud database (not shown in the figure). The cloud database used in the user learning pattern detection system 100 is AWS S3. Although for storing the data, the user learning pattern detection system 100 is not limited to the AWS S3, it may use other tools like Google Cloud Storage, Azure Blob Storage, iCloud, Microsoft Azure, IBM Cloud, and so on.

The output that is stored in the cloud database in JSON format is given below:

### JSON Schema:

```
{
  “$schema”: “http://json-schema.org/draft-07/schema#”,
  “type”: “object”,
  “properties”: {
    “student_email”: {
      “type”: “string”,
      “format”: “email”
    },
    “date”: {
      “type”: “string”,
      “format”: “date”
    },
    “sessions”: {
      “type”: “array”,
      “items”: {
        “type”: “object”,
        “properties”: {
          “session_id”: {
            “type”: “string”
          },
          “anti_patterns”: {
            “type”: “array”,
            “items”: {
              “type”: “object”,
              “properties”: {
                “antipattern_id”: {
                  “type”: “string”
                },
                “name”: {
                  “type”: “string”
                },
                “description”: {
                  “type”: “string”
                },
                “app”: {
                  “type”: “string”
                },
                “subject”: {
                  “type”: “string”
                },
                “clip_start_time”: {
                  “type”: “integer”
                },
                “clip_end_time”: {
                  “type”: “integer”
                },
                “clip_length”: {
                  “type”: “integer”
                },
                “trimmed_clip_link”: {
                  “type”: “string”,
                  “format”: “uri”
                },
                “evidence”: {
                  “type”: “string”
                },
                “session_id”: {
                  “type”: “string”
                },
                  “student_email”: {
                  “type”: “string”,
                  “format”: “email”
                }
              },
              “required”: [“antipattern_id”, “name”, “description”, “app”,
“subject”, “clip_start_time”, “clip_end_time”, “clip_length”,
“trimmed_clip_link”, “evidence”, “session_id”, “student_email”]
            }
          },
          “status”: {
              “type”: “string”
            }
          },
          “required”: [“session_id”, “anti_patterns”, “status”]
        }
      },
      “processed”: {
        “type”: “boolean”
      }
    },
    “required”: [“student_email”, “date”, “sessions”, “processed”]
  }
Example:
    {
            “student_email”: “lucian.klinefelter@alpha.school”,
            “date”: “2024-02-29”,
            “sessions”: [
                {
                    “session_id”: “539526”,
                    “anti_patterns”: [
                        {
                            “antipattern_id”: “4”,
                            “name”: “ANTI | ONLY ignoring explanations after
    mistakes (NOT rushing/guessing questions)”,
                            “description”: “Despite not rushing through the
    question, at the end the student skips the in-depth explanations provided
    by the app for incorrect answers, missing out on learning from their
    mistakes.”,
                            “app”: “IXL”,
                            “subject”: “Language”,
                            “clip_start_time”: 1387,
                            “clip_end_time”: 1398,
                            “clip_length”: 11,
                            “trimmed_clip_link”: “https://studyreel-ai-
    clips.s3.us-east-1.amazonaws.com/2024-03-
    01/539526/APnotrushingquestionrushingexplanation/521f3d4f4ed5409486d00a43f
    1c9937a.mp4”,
                            “evidence”: “Detected anti-pattern at 00:23:07
    with question duration 00:01:23 and explanation duration 00:00:04.”,
                            “session_id”: “539526”,
                            “student_email”: “lucian.klinefelter@alpha.school”
                        }
                    ],
                    “status”: “PROCESSED”
                },
                {
                    “session_id”: “539394”,
                    “anti_patterns”: [],
                    “status”: “PROCESSED”
                }
            ],
            “processed”: true
    }
```

This code is a JSON schema that defines the structure and validation rules for a JSON object used to store and process information about the user's online learning sessions. The schema specifies that the main object must include several required properties, such as the student's email, the date of the sessions, an array of session details, and a boolean flag indicating whether the data has been processed.

The student_email is defined as a string that must follow an email format. The date property is also a string but must follow the date format. The sessions property is an array containing multiple session objects, where each session represents an individual learning session the student participated in.

Each session object must include a session_id as a string, an array of anti_patterns, and a status string indicating the session's processing state. The anti_patterns array within each session contains objects that describe specific undesirable behaviors or patterns detected during the online learning session. These anti-pattern objects include an antipattern_id, a name and description of the anti-pattern, the app and subject related to the anti-pattern, and the timestamps (clip_start_time, clip_end_time, and clip_length) marking where the anti-pattern occurred in the session video clip 138. Additionally, the anti-pattern object includes a trimmed_clip_link, which is a URI pointing to video clip 138 showing the anti-pattern, and an evidence string providing a summary of the detected behavior.

The following pseudo-code is used to manage and prompt AI ENGINE 128 of the user learning pattern detection system 100 to detect anti-patterns/posi-patterns:

```
# Import necessary machine learning, computer vision, and data analytics
utils and libraries
import machine_learning_utils as ml
import computer_vision_library as cv
import data_analytics_util as da
# Define the main class for the Antipattern Detection System
class AntipatternDetectionSystem:
    def __init__(self, session):
        # Initialize the AI models for video analysis and qc engine
        self.video_analysis_model =
ml.load_model(‘video_analysis_model path’)
        # Initialize the AI model for quality control
        self.quality_control_model =
ml.load_model (‘quality_control_model_path’)
        # Initialize the data analytics util
        self.data_analysis_tool = da.load_util(session.id)
    def analyze_student_behavior(self, video_data, performance_data):
        ″″″
        Analyze student behavior by integrating video,, and real time
performance data.
        ″″″
        # Analyze educational performance data to detect learning antipatterns
        performance_antipatterns =
self.data_analysis_tool.detect_antipatterns(performance_data)
        # Analyze video data to detect visual antipatterns
        video_antipatterns =
self.video_analysis_model.detect_antipatterns(video_data)
        # Integrate multimodal data for comprehensive analysis
        integrated_analysis = self.integrate_data(video_antipatterns,
performance_antipatterns)
        # Perform AI-driven quality control to validate the detection results
        quality_control_results =
self.quality_control_model.validate_detection(integrated_analysis)
        # Return the final analysis results after quality control
        return quality_control_results
    def integrate_data(self, video_antipatterns,, performance_antipatterns):
        ″″″
        Integrate video and performance antipatterns for a holistic view.
        ″″″
        # Combine the antipatterns detected from different modalities
        combined_antipatterns = da.combine_data(video_antipatterns,
performance_antipatterns)
        # Analyze the combined data to identify correlations and insights
        integrated_analysis = da.analyze_combined_data(combined_antipatterns)
        return integrated_analysis
# Instantiate the system
antipattern_detection_system = AntipatternDetectionSystem( )
# Example usage of the system with dummy data
final_results =
antipattern_detection_system.analyze_student_behavior(video_data=‘video_strea
m’, performance_data=‘student_performance_metrics’)
```

In an embodiment of the user learning pattern detection system 100, the engagement score of the user is calculated. The calculation of the user's engagement score begins with detecting user engagement, which is achieved by analyzing video recordings captured from the user's webcam. The analysis focuses on key visual indicators that reflect the user's level of engagement, such as maintaining eye contact with the screen, interpreting facial expressions that might indicate interest or distraction, and observing body posture that could signal attentiveness or disengagement. By closely monitoring these indicators, analyzer 122 can assess how actively the user participates in the learning session.

Once the visual data is analyzed, the AI engine 128 calculates an engagement score, which measures the level of user engagement based on the observed behaviors. This score is derived from the patterns detected in the user's learning behaviors, providing a measurable metric that reflects the user's attention and involvement during the online learning session. The engagement score is then used to assess the overall participation of the user. By tracking these scores over time, the AI engine 128 can identify patterns of consistent engagement or signs of disengagement. This allows educators to monitor the user's learning experience more effectively and intervene when necessary to enhance the learning outcomes, such as by adjusting the teaching approach or providing additional support.

The user learning pattern detection system 100 also establishes threshold values for specific indicators of the user's learning behavior. These thresholds serve as benchmarks for acceptable levels of engagement, gaze direction, and the frequency of anti-pattern occurrences, such as distractions or lapses in attention. The detected behavior metrics are then compared against these thresholds to determine whether the user's behavior falls within acceptable ranges. If the metrics exceed or fall below the established thresholds, indicating potential issues with the user's engagement or learning habits, the user learning pattern detection system 100 provides recommendations to address these concerns. For instance, if the user's engagement levels are consistently low, the user learning pattern detection system 100 might suggest strategies to improve focus and participation, thereby helping to maintain or improve the user's learning.

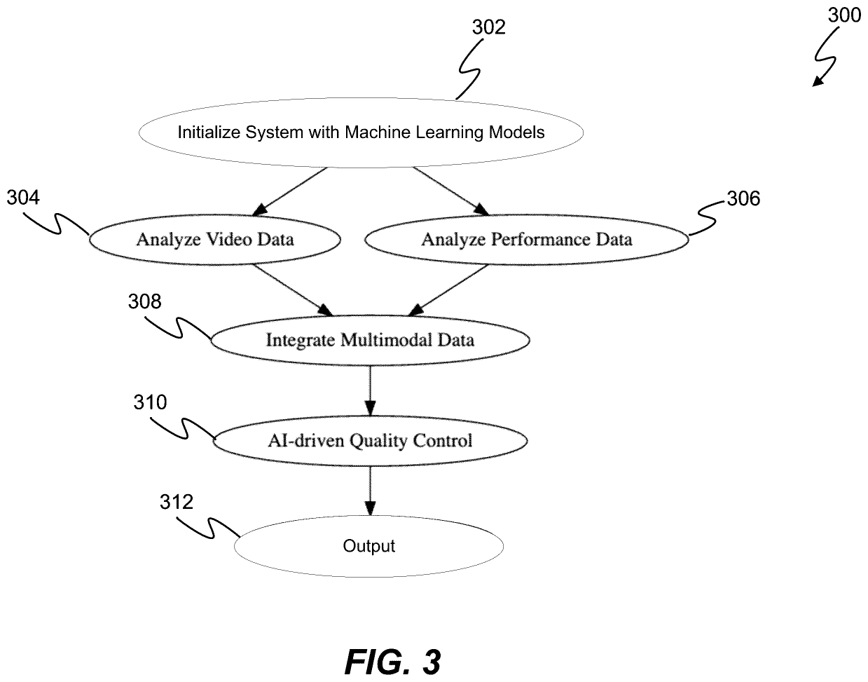

FIG. 3 depicts an exemplary report and video clips featuring the user learning patterns generation process 300, which is an embodiment of the user learning pattern detection process 200 based on media stream data 116 and user data analytics of FIG. 2.

The report and video clips featuring the user learning patterns generation process 300 begins by initializing the AI mode 302, like multimodal LLM, which activates the AI components like AI engine 128, which is responsible for detecting the anti-patterns or posi-patterns of the user during the online learning session.

Once the AI model is initialized 302, it proceeds to analyze the video data 304 by utilizing the analyzer 122. This step involves analyzing the content of the video, such as monitoring user interactions during an online learning session, identifying key events, and detecting potential patterns in the user's behavior. For example, the analyzer 122 might track how often a user rewinds the video, pauses it, or skips certain sections, which could indicate areas of difficulty or lack of interest.

Following the video analysis, the analyzer 122 shifts focus to performance data 306 to assess how the user is performing during the online learning session. This might include evaluating quiz results, tracking the speed at which the user progresses through the material, and noting any instances of distraction. Next, the multimodal data 308 is integrated by combining information from different sources such as video content, performance metrics, and possibly biometric data like eye movement or facial expressions to form a comprehensive understanding of the user's behavior.

The AI-driven quality control process 310 utilizes the quality checker 136 ensuring that all analyzed data meets predefined standards of accuracy and relevance. This step verifies that the conclusions drawn by the AI engine 128 are based on reliable data, free from errors or inconsistencies. For example, the quality control might ensure that the video analysis correctly identifies all instances of user interaction and that the performance data reflects true user engagement without technical issues.

Finally, the AI engine 128 outputs the final results 312, which include video clips 138 that feature moments from the online learning session where an antipattern or posi-pattern occurred. These clips are accompanied by detailed reports and recommendations which are generated in correspondence to the user's behavior. For instance, if the AI engine 128 detects that a user is getting distracted frequently, the report might include advice on minimizing distractions, while a praise message might be generated if the user is consistently focused. The report also provides specifics such as the duration of each detected antipattern or posi-pattern, and it includes hyperlinks allowing the user to access the relevant video clips 138 for review. This comprehensive output ensures that users receive actionable feedback, helping them to improve their learning experience.

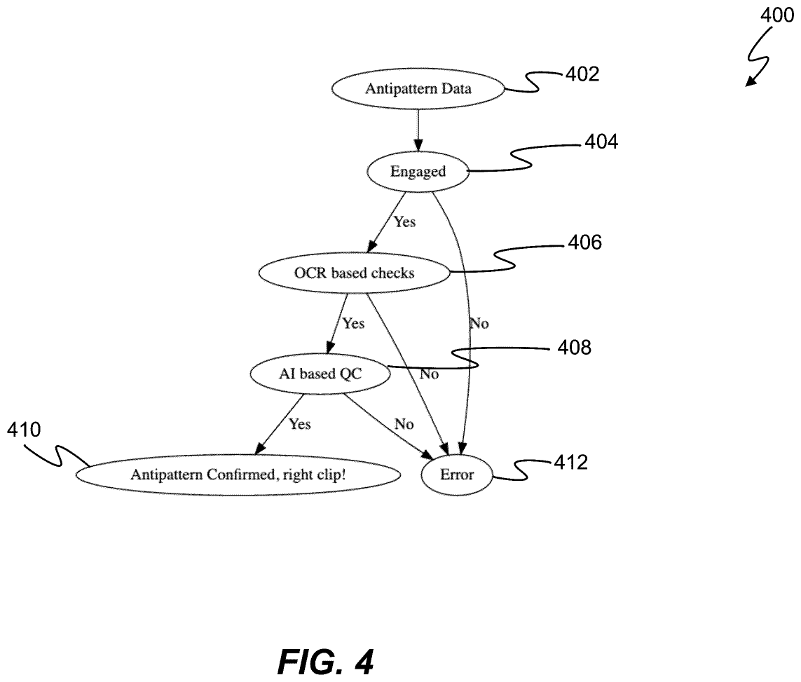

FIG. 4 depicts an exemplary anti-patterns quality check process 400, which is an embodiment of the user learning pattern detection process 200 based on media stream data 116 and user data analytics of FIG. 2.

The anti-patterns quality check process 400 begins with the detection of antipattern data by the learning pattern detector 132, which identifies potential deviations from expected patterns 402. For instance, if a user opens some external apps during the online learning session, if the user scrolls the content of the webpage very quickly etc., the learning pattern detector 132 might flag this as anti-pattern behavior. Once detected, this data is passed to the classifier 134, which categorizes it into either an antipattern or a posi-pattern. Antipatterns represent undesirable or inefficient behaviors, while posi-patterns reflect positive and efficient usage patterns.

Following the classification, the user engagement is checked 404. Engagement could involve monitoring if the user is interacting with the content or showing signs of focus, such as consistent viewing without excessive pauses. For example, a user who watches a video without skipping might be considered engaged, and a user taking more than the predefined threshold time to complete the task provided during the online learning session.

After confirming engagement, the quality checker 136 performs OCR-based checks 406 to analyze text within the video or any displayed content. These checks might involve verifying that subtitles match the spoken content or ensuring that on-screen text is free of errors. For example, if the video contains a slide presentation, the OCR check would confirm that the text on the slides is legible and accurate.

Next, the AI-driven quality checker 136 performs a quality control QC check 408 to validate the overall integrity of the content. This step involves assessing the video's technical quality, such as ensuring there are no glitches, verifying that the audio syncs with the video, and confirming that the video adheres to predefined quality standards. For example, the QC check might detect that the video resolution drops unexpectedly, prompting a review.

If the antipattern data is confirmed through these checks 410, the right video clip 138 is then assessed thoroughly and classified under the antipattern category. This means the system has identified and validated the behavior or content as being problematic, such as repeatedly skipping critical content sections.

However, if the detected video clip 136 contains errors 412 and is not classified as an antipattern, this might indicate that issues occurred during the detection process. For instance, errors could arise from a network problem leading to incomplete data transmission, or a recording error might result in incorrect timestamps, causing the system to misinterpret user behavior or video content. Such errors would lead the system to reevaluate the clip, potentially classifying it as non-antipattern if the issues are resolved.

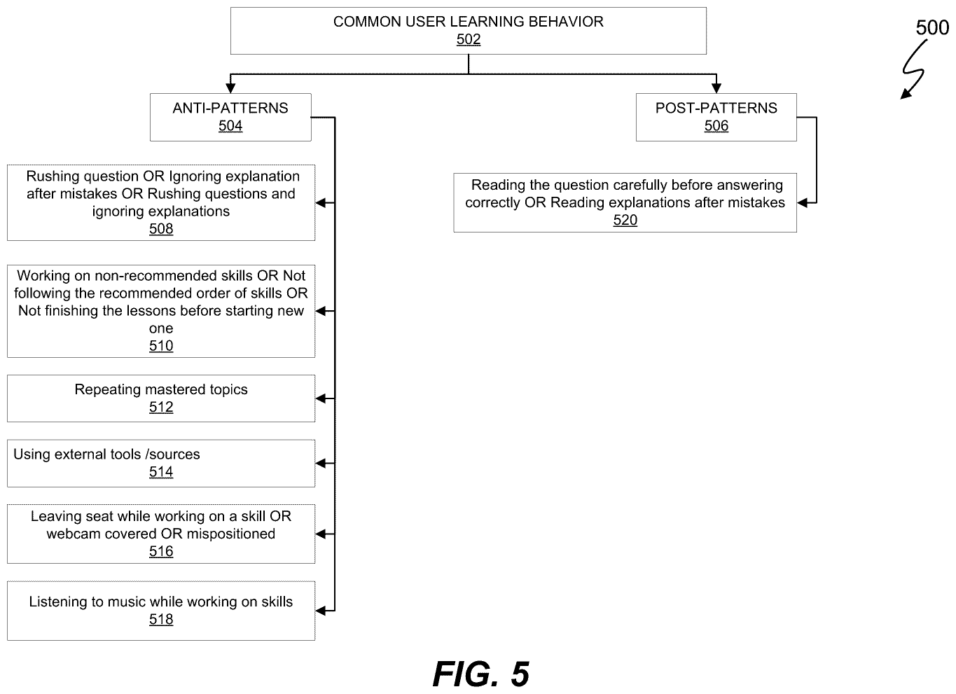

FIG. 5 depicts the common user learning patterns 502 which are taken into consideration by the user learning pattern detection system 100 based on media stream data 116 and user data analytics.

The common user learning patterns 502 are discussed in detail in FIG. 5. The learning patterns of the user are detected by the learning pattern detector 132, which utilizes AI NLP techniques, as discussed in detail in FIGS. 1 and 2. Upon detection of the learning patterns of the user, the classifier 134 classifies the learning patterns of the user into anti-patterns 504 and psoi-patterns 506.

Anti-patterns 504 are recurrent behaviors, strategies, or practices that are counterproductive to learning. These are habits that, when consistently followed, tend to hinder the learning of the user. On the other hand, posi-patterns are behaviors, strategies, or practices that are conducive to effective learning. These patterns promote better understanding and retention. The common user learning patterns 502 which are utilized in the user learning pattern detection system 100 are explained below in detail.

The antipatterns Rushing questions, Ignoring explanations after mistakes, and Rushing questions and ignoring explanations 508 focus on identifying behaviors where users may not be fully engaging with their learning tasks. These antipatterns rely primarily on data from the user engagement data 114 stored in the data collector 120, which provides detailed information on questions and explanations. For each online learning session, the data from the online learning platforms 104 are queried to retrieve the relevant data, including question and explanation durations.

For apps not supported by the learning pattern analysis module 118, raw request events are used to gather this data. This is done by filtering out questions that don't meet specific criteria, such as minimum accuracy or whether an explanation is provided. If the duration of the question or the explanation is below a certain threshold, the event is flagged as an antipattern. Once an antipattern is identified, a start and end window are calculated around this event using predefined attributes. Antipatterns that occur too close to the session's start or end are discarded.

After identification, each antipattern event undergoes further processing to exclude those that happen during black screen time, ensuring the accuracy of the analysis. For these antipatterns, the user's webcam status is not considered, so no distraction checks are included. Exceptions include dropping antipatterns that occur within 20 seconds of the session's start or end. Specifically for Ignoring explanations, for instance, if the app is Khan Academy and the explanation duration is 2 seconds or less, the event is discarded to avoid false positives from bad clips previously observed.

The antipatterns ‘Working on non-recommended skills, Not following the recommended order of skills, and Not finishing a lesson before starting a new one’ 510 focus on monitoring whether users follow their recommended learning paths. For Working on non-recommended skills, the data collector 120 fetches course recommendations from the API 140, and the analyzer 122 checks if each quiz URL matches the recommended skills for the session. If a quiz does not align with the recommendations, it is flagged as an antipattern by the learning pattern detector 132.

Not following the recommended order of skills identifies when a user skips ahead in their learning sequence. The learning pattern detector 132 checks if a quiz is part of the recommended order and if any prior recommendations in the same subject are incomplete (indicated by a smart score of 0), the quiz session is marked as an antipattern.

Similarly, Not finishing a lesson before starting a new one flag instances where a user begins a new lesson without completing the previous one. This is determined by checking if the current quiz matches a recommendation and if earlier lessons have a smart score between 0 and 100, indicating partial completion.

For all three antipatterns ‘Working on non-recommended skills, Not following the recommended order of skills, and Not finishing a lesson before starting a new one’ 510, any events occurring within 20 seconds of the session's start or end are excluded. Additionally, if no question attempt results in at least seven minutes of engagement, clips are shortened, and quiz attempts are cross-referenced with Coachbot data. Events with very short detection lengths or incorrect URL sequences are also discarded, ensuring that only relevant and accurate instances are flagged.

The antipattern ‘Repeating mastered topics’ 512 identifies instances where a user revisits quizzes they have already mastered. The list of mastered quizzes is fetched by the data collector 120 from the user engagement data 114. The date and time of each online learning session is fetched to get the unique names of these mastered quizzes. Then, using the details received from API 140, analyzer 122 checks the user's learning recommendations for that online learning session date to ensure the recommendations were made before or on the online learning session date and were completed or invalidated afterward.

During each online learning session, if the user attempts a quiz that was recommended to them, it is not considered an antipattern. However, if the attempted quiz is part of the mastered list, the time spent on it is marked as an antipattern instance. The learning pattern detector 132 excludes antipatterns that occur during black screen periods or within 20 seconds of the start or end of a session. The antipattern instances are also adjusted by shortening clips to four minutes if no question attempt results in seven minutes of behavior. Special handling is applied for different platforms like IXL, AlphaRead, and KhanAcademy based on quiz URLs. Question attempts for IXL and AlphaRead are checked per quiz, and matched with data collected by converting the quiz data into quiz URLs. For KhanAcademy, if the quiz URL is collected by the data collector, checking is done per quiz. If the quiz URL is not present, checking is done with the subject matched. Notably, a user's webcam being turned on is not required for this antipattern, so no distraction checks are included.

The antipattern ‘Using external tools/sources’ 514 is particularly designed for some online learning platforms like Austin Speedrun Contest and Edulastic. The Austin Private Schools SPEEDRUN Contest is an exciting and challenging competition designed for students of Austin's private schools. This contest aims to encourage and recognize excellence in mathematics among students. The contest is structured to test students' mastery of essential math lessons through a competitive and rewarding format. Edulastic is a web-based platform that helps teachers and school administrators create and monitor online assessments for K-12 students. It provides teachers with real-time classroom data to help them identify students who are on track and who need help. Edulastic was launched in June 2014 by Snapwiz, an education technology company.

Whenever the user accesses any external URLs, the screen image is captured and stored in the media stream data 116, which when analyzed by the analyzer 122, is classified as the anti-pattern by the learning pattern detector 132. In the case of the user learning pattern detection system 100, the URL of the online learning session is checked against a whitelist. If a non-whitelisted URL is visited, the segment is flagged as an antipattern.

Further, in the case of the Edulastic application, only practice and assessment URLs are considered valid, and any access to external URLs during these sessions is marked as an antipattern instance.

The antipatterns ‘Leaving seat while working on a skill and Webcam covered or mispositioned’ 516 utilizes Mediapipe's face detection model to identify when the user is disengaged during the online learning session. The MediaPipe Face Detector task detects faces in an image or video. It can be used to locate faces and facial features within a frame. It utilizes a machine learning (ML) model that works with single images or a continuous stream of images. Although the user learning patterns detection system 100 is not limited to MediaPipe Face Detector, it may also include Blaze Face, Retina Face, and so on.

For Leaving Seat, the learning pattern detector 132 checks if the user is actively working on a skill. If no face is detected for over 30 seconds, the learning pattern detector 132 marks this period as the anti-pattern. For Webcam covered or mispositioned, the algorithm detects if the face is missing while ensuring the user is not idle and hasn't triggered the Leaving Seat antipattern. If these conditions are met, the absence of a face is flagged as an antipattern.

The final anti-pattern 504 includes ‘Listening to music while working on skills’ 518. The media stream data 116 utilizes microphone data, as well as the data of the websites, along with the URLs surfed by the user. This data when analyzed by the analyzer 122 helps the learning pattern detector 132 to detect the anti-pattern ‘Listening to music while working on skills’ 518.

The posi-pattern 506 includes ‘Reading the question carefully before answering correctly’ or ‘Reading explanations after mistakes’ 518. The posi-patterns ‘Reading the question carefully before answering correctly and Reading explanations after mistakes’ 520 aim to assess user engagement by tracking specific behaviors during online learning sessions.

After the analysis of the collected data using the analyzer 122, the AI engine 128 utilizes the learning pattern detector 132 to detect the learning patterns of the user. The classifier 134 classifies the learning patterns into anti-pattern 504 and posi-pattern 506. The learning pattern detector 132 identifies and calculates relevant time windows, each posi-pattern 506 is further scrutinized to exclude instances occurring during black screen periods, with an additional requirement that the user's webcam must be on. This ensures that the user is engaged throughout the POSI event. If an anti-pattern occurs within 20 seconds of the start or end of the online learning session, it is discarded to maintain accuracy.

The Engagement Analyzer algorithm, which processes videos to identify engagement, begins by checking whether a frame has been analyzed and then processes frames to detect faces and facial details. The processing of frames to detect faces is performed using AWS Rekognition. Amazon Rekognition is a cloud-based image and video analysis service that makes it easy to add advanced computer vision capabilities to your applications. Although the user learning pattern detection system 100 is not limited to AWS recognition, other similar tools for face detection like Google Cloud Vision, Microsoft Azure Custom Vision, Open CV, Azure Face, and so on can also be used.

The algorithm flags frames with multiple faces, low confidence in detection, or if the user's gaze is away from the screen. This information is stored and used to mark engagement or distraction.

The following pseudo-code is used to manage and prompt AI ENGINE 128 of the user learning pattern detection system 100 for analyzing user engagement during the online learning session:

### Algorithm for Engagement Analyzer:

1. Initialize EngagementAnalyzer with video path and second interval.
2. Loop over the AntiPatterns for the given video:
   2.1. If the AntiPattern start timestamp is already present in the frame markers map, it means this frame has already been analyzed, so skip it and move to the next AntiPattern.
   2.2. If the AntiPattern start timestamp is not present in the frame markers map, read the corresponding frame from the video.
   2.3. Crop the frame to focus on the webcam feed and convert it into bytes.
   2.4. Send the byte data to AWS Recognition service to detect faces and determine facial details, which include eyes direction, eyes status, and age range.
   2.5. Check the number of faces detected. If there is more than one face or if the detected face is of a guide (determined based on a age threshold), mark the frame as having multiple faces or a guide present. Include this information in the frame markers map for this AntiPattern timestamp and move on to the next AntiPattern.
   2.6. If only one face that is not a guide is detected, check the confidence level of the detection. If it's below a certain threshold (indicating low confidence in the detection), tag this frame as distracted. Store this finding in the frame markers map for this AntiPattern start timestamp and move on to the next AntiPattern.
   2.7. With a high confidence detection, utilize the Eye Direction attribute of AWS Rekognition service response, check if it exists.
      If it doesn't exist, mark the frame as distracted.
      If it does exist, evaluate if the person is looking away from the screen by checking eye direction coordinates (pitch and yaw) against thresholds. If the eye movement is away from the screen beyond the thresholds, mark the frame as distracted.
   2.8. Store this detailed information for the frame in the frame markers map with the AntiPattern's start timestamp as the key.
3. Continue this process for all the AntiPatterns in the video for their whole duration, advancing the input second interval at a time.
4. Repeat the process of engagement detection for all AntiPatterns in the video.

Additionally, for the posi-pattern ‘Reading explanations after mistakes’ 520, a ScrollDetector algorithm checks if users fully read explanations by monitoring scrolling activity. The ScrollDetector algorithm assesses whether the content on the online learning platform 104, say, IXL page is scrollable, whether scrolling occurs, and whether the end of the content is reached by the user or not. IXL is a personalized learning program that offers practice modules for grades Pre-K-12 in subjects like math, English, science, social studies, and Spanish. IXL is available on the web, as well as on Android, iPod Touch, iPhone, and iPad.

This data is stored to verify that users are not just skipping but thoroughly engaging with the material. For this purpose, in applications like IXL, a guide button is provided at the end of the page. When the user completes the whole page by scrolling through the whole page, the user has to click on that guide button to go to the next page. Further, there is a threshold time defined for the scrolling of the content of the page. If the user comes to the end of the page, by scrolling fastly without meeting the threshold requirements, then the timestamp will be recorded and it will be considered as anti-pattern.

The following pseudo-code is used to manage and prompt AI ENGINE 128 of the user learning pattern detection system 100 for scroll detection:

### Algorithm for ScrollDetector:

1. Initialize ScrollDetector with video path and recording session data
2. For each AntiPattern in the analyzed AntiPattern list:
   2.1 Check if the application used is supported. If not supported, continue to next AntiPattern.
   2.2 Determine the start and end frames of the AntiPattern segment in the video.
   2.3 Calculate the frames to check for scrolling within the AntiPattern segment using the start and end frames.
   2.4 Extract OCR data from the first frame and check if the content is scrollable (is content scrollable). Store this is content scrollable information for the AntiPattern.
   2.5 With the OCR data for each frame, check two things: If scrolling occurred (scroll states) & if end of content is reached (end reached).
   2.6 If the content is scrollable, compute the percentage of frames in which scrolling occurred (scroll perc).
   2.7 Store the above collected information in the QC checks of the AntiPattern.
Output: Updated list of AntiPatterns with information about scrollability and occurrence of scrolling for each AntiPattern.

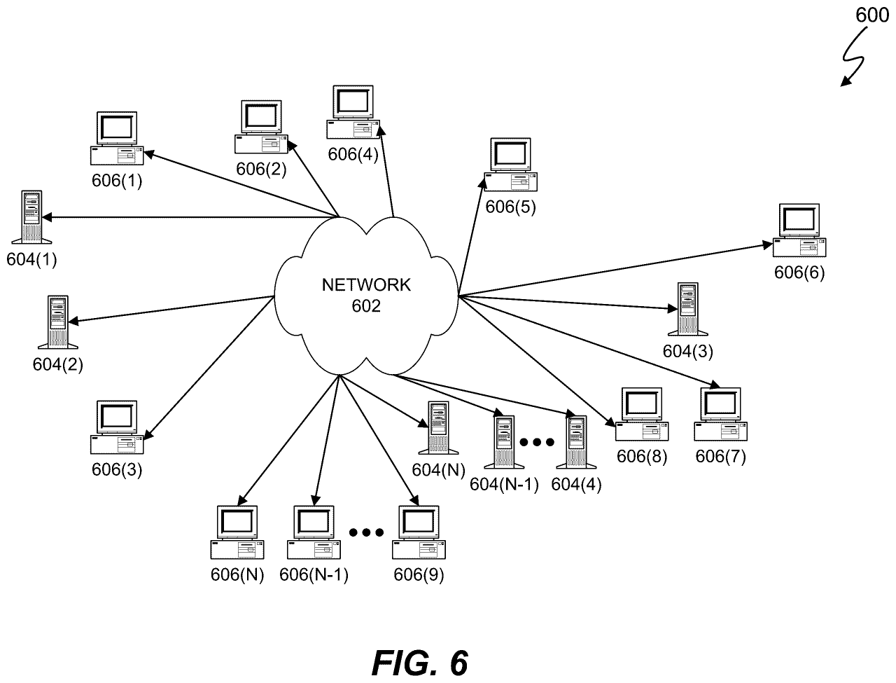

FIG. 6 is a block diagram illustrating a network environment in which a user learning pattern detection system 100 and process 200 based on media stream data 116 and user data analytics may be practiced. Network 602 (e.g. a private wide area network (WAN) or the Internet) includes several networked server computer systems 604(1)-(N) that are accessible by client computer systems 606(1)-(N), where N is the number of server computer systems connected to the network. Communication between client computer systems 606(1)-(N) and server computer systems 604(1)-(N) typically occurs over a network, such as a public switched telephone network over asynchronous digital subscriber line (ADSL) telephone lines or high-bandwidth trunks, for example, communications channels providing T1 or OC3 service. Client computer systems 606(1)-(N) typically access server computer systems 604(1)-(N) through a service provider, such as an internet service provider (“ISP”) by executing application-specific software, commonly referred to as a browser, on one of client computer systems 606(1)-(N).

Client computer systems 606(1)-(N) and/or server computer systems 604(1)-(N) are specialized computers programmed to improve conventional computer systems to implement and utilize the user learning pattern detection system 100 and process 200 based on media stream data 116 and user data analytics. The type of computer system that can be specially programmed to implement and utilize the user learning pattern detection system 100 and process 200 based on media stream data 116 and user data analytics includes a mainframe, a mini-computer, a personal computer system including notebook computers, a wireless, mobile computing device (including personal digital assistants, smartphones, and tablet computers). These computer systems are typically designed to provide computing power to one or more users, either locally or remotely. Each computer system may also include one or a plurality of input/output (“I/O”) devices coupled to the system processor to perform specialized functions. Tangible, non-transitory memories (also referred to as “storage devices”) such as hard disks, compact disk (“CD”) drives, digital versatile disk (“DVD”) drives, and magneto-optical drives may also be provided, either as an integrated or peripheral device. In at least one embodiment, the user learning pattern detection system 100 and process 200 based on media stream data 116 and user data analytics can be implemented using code stored in a tangible, non-transient computer-readable medium and executed by one or more processors. In at least one embodiment, the user learning pattern detection system 100 and process 200 based on media stream data 116 and user data analytics can be implemented completely in hardware using, for example, logic circuits and other circuits including field programmable gate arrays.

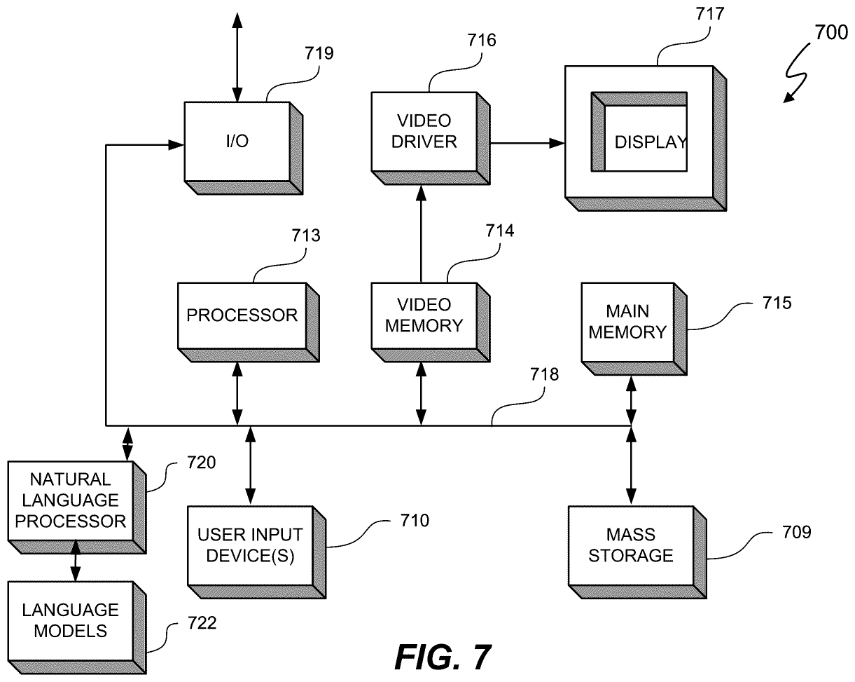

Embodiments of the user learning pattern detection system 100 and process 200 based on media stream data 116 and user data analytics can be implemented on a computer system such as a special-purpose, special-programmed computer 700 illustrated in FIG. 7. The input user device(s) 710, such as a keyboard and/or mouse, are coupled to a bi-directional system bus 718. The input user device(s) 710 are for introducing user input to the computer system and communicating that user input to the processor 713. The computer system of FIG. 7 generally also includes a non-transitory video memory 714, non-transitory main memory 715, and non-transitory mass storage 709, all coupled to the bi-directional system bus 718 along with input user device(s) 710 and processor 713. The mass storage 709 may include both fixed and removable media, such as a hard drive, one or more CDs or DVDs, solid state memory including flash memory, and other available mass storage technology. Bus 718 may contain, for example, 32 of 64 address lines for addressing video memory 714 or main memory 715. The system bus 718 also includes, for example, an n-bit data bus for transferring DATA between and among the components, such as CPU 709, main memory 715, video memory 714, and mass storage 709, where “n” is, for example, 32 or 64. Alternatively, multiplex data/address lines may be used instead of separate data and address lines.

I/O device(s) 719 may provide connections to peripheral devices, such as a printer, and may also provide a direct connection to a remote server computer system via a telephone link or to the Internet via an ISP. I/O device(s) 719 may also include a network interface device to provide a direct connection to a remote server computer system via a direct network link to the Internet via a POP (point of presence). Such connection may be made using, for example, wireless techniques, including digital cellular telephone connection, Cellular Digital Packet Data (CDPD) connection, digital satellite data connection, or the like. Examples of I/O devices include modems, sound and video devices, and specialized communication devices such as the aforementioned network interface.

Computer programs and data are generally stored as code in a non-transient computer-readable medium such as flash memory, optical memory, magnetic memory, compact disks, digital versatile disks, and any other type of memory. The computer program is loaded from a memory, such as mass storage 709, into main memory 715 for execution. Computer programs may also be in the form of electronic signals modulated in accordance with the computer program and data communication technology when transferred via a network. In at least one embodiment, Java applets or any other technology is used with web pages to allow a user of a web browser to make and submit selections and allow a client computer system to capture the user selection and submit the selection data to a server computer system.

The processor 713, in one embodiment, is a microprocessor manufactured by Motorola Inc. of Illinois, Intel Corporation of California, or Advanced Micro Devices of California. However, any other suitable single or multiple microprocessors or microcomputers may be utilized. Main memory 715 consists of dynamic random access memory (DRAM). Video memory 714 is a dual-ported video random access memory. One port of the video memory 714 is coupled to the video amplifier 716. The video amplifier 716 is used to drive the display 717. Video amplifier 716 is well-known in the art and may be implemented by any suitable means. This circuitry converts pixel DATA stored in video memory 714 to a raster signal suitable for use by display 717. Display 717 is a type of monitor suitable for displaying graphic images.

The computer system described above is for purposes of example only. The user learning pattern detection system 100 and process 200 based on media stream data 116 and user data analytics may be implemented in any type of computer system or programming or processing environment. It is contemplated that the user learning pattern detection system 100 and process 200 based on media stream data 116 and user data analytics might be run on a stand-alone computer system, such as the one described above. The user learning pattern detection system 100 and process 200 based on media stream data 116 and user data analytics might also be run from a server computer system that can be accessed by a plurality of client computer systems interconnected over an intranet network. Finally, the user learning pattern detection system 100 and process 200 based on media stream data 116, and user data analytics may be run from a server computer system that is accessible to clients over the Internet.

Although embodiments have been described in detail, it should be understood that various changes, substitutions, and alterations can be made herein without departing from the spirit and scope of the invention as defined by the appended claims.

What is claimed is:

1. A method of guiding an Artificial Intelligence (AI) engine to identify and analyze an anti-pattern or a posi-pattern when a user is using an online learning application, the method comprises:
executing code using one or more processors of a computer system to cause the computer system to perform operations comprising:
collecting media stream data, user interaction data, and user engagement data, wherein the media stream data includes webcam feed, microphone audio, screen captures, and system audio, and user interaction data includes keystrokes, mouse clicks, URLs visited, active application data, active window data, and window titles;
analyzing the pre-processed data to generate insights that indicate the learning behavior of the user using the online learning platform;
guiding and constraining an AI engine to perform operations comprising:
detecting the learning behaviors of the user using the online learning platform using machine learning algorithms and computer vision techniques;
classifying the detected user's learning behavior into positive learning patterns (posi-patterns) and negative learning patterns (anti-patterns);
performing a quality check on the detected anti-patterns and posi-patterns using multimodal large language models (LLMs), to verify the accuracy and relevance of the detected patterns;
generating reports that verify anti-patterns and posi-patterns and provide recommendations to the user, wherein the reports include a video clip featuring the section where anti-pattern or posi-patterns occurred.

2. The method of claim 1 wherein the media stream data is collected using a microphone or webcam that may be either integrated within the user's device or operatively coupled to the user's device.

3. The method of claim 1 wherein the media stream data include both webcam footage and screen activity of the user, allowing for a comprehensive assessment of engagement with educational content.

4. The method of claim 1 wherein the user engagement data includes the user's browsing history, test scores, assignment completion rates, and time spent on specific tasks.

5. The method of claim 1 further comprises:
collecting the data and time of the question asked during the online learning session, and the quiz details, including the time taken to attempt the quiz, and correct and incorrect answers.

6. The method of claim 1 further comprises:
pre-processing the collected data to organize it in a structured format, wherein the structured format includes clear and formatted data that is ready for analysis.

7. The method of claim 1 wherein the analyzed insights helps in prompt generation by populating the prompt structure provided by the prompt engineer.

8. The method of claim 1 further comprises:
utilizing gaze detection techniques to monitor the direction of a user's gaze while interacting with content on the online learning platform;
analyzing the gaze data to determine the extent to which the user has visually engaged with the content, including determining whether the user is focusing on the content or looking away from the screen;
incorporating the gaze data into the overall analysis of the user's learning behavior, thereby identifying potential anti-patterns such as distraction or lack of focus, or posi-patterns such as sustained attention.

9. The method of claim 1 further comprises:
selecting specific segments of video recordings that visually demonstrate the identified patterns, such as clips showing instances of user distraction (anti-patterns) or active participation (posi-patterns);
generating video clips that highlight the relevant anti-patterns and posi-patterns detected in the user's learning behaviors;
providing the generated video clips to educators along with analysis reports that explain the context of the patterns observed, allowing educators to visually review the evidence of student behaviors and apply the insights to improve teaching strategies.

10. The method of claim 1 wherein the AI engine utilizes a Vision Large Language Model (LLM-V) capable of interpreting and understanding images paired with text, enabling the AI engine to process and analyze multimodal data.

11. The method of claim 1 further comprises:
detecting user engagement scores by analyzing video recordings from the user's webcam, wherein the analysis includes assessing visual indicators of engagement such as eye contact with the screen, facial expressions, and body posture;
calculating the level of user engagement during online learning sessions by assigning an engagement score based on the observed user learning behaviors;
utilizing the engagement scores to assess overall user participation and identify patterns of engagement or disengagement, thereby enabling educators to intervene when necessary to improve the user's learning outcomes.

12. The method of claim 1 further comprises:
establishing threshold values for specific indicators of the user's learning behavior, including but not limited to engagement levels, gaze direction, and frequency of anti-pattern occurrences;
comparing the detected behavior metrics against the established threshold values to determine whether the behavior falls within acceptable ranges;
providing recommendations when the user's learning behavior metrics exceed or fall below the threshold values.

13. The method of claim 1 wherein the recommendations address specific behaviors detected during the user's online learning session and suggest corrective actions to improve learning efficiency.

14. A system to guide an Artificial Intelligence (AI) engine to identify and analyze an anti-pattern or posi-pattern when a user is using an online learning application comprises:
one or more processors;
a memory, coupled to the one or more processors, storing code that when executed by the one or more processors cause a computer system to perform operations comprising:
collecting media stream data, user interaction data, and user engagement data using a data collector, wherein the media stream data includes webcam feed, microphone audio, screen captures, and system audio, and user interaction data includes keystrokes, mouse clicks, URLs visited, active application data, active window data, and window titles;
analyzing the collected data using an analyzer to generate insights that indicate the learning behavior of the user using the online learning platform;
guiding and constraining an AI engine to perform operations comprising:
detecting the learning behaviors of the user using the online learning platform using a learning pattern detector that utilizes machine learning algorithms and computer vision techniques;
classifying the detected user's learning behavior into positive learning patterns (posi-patterns) and negative learning patterns (anti-patterns) using a classifier;
performing a quality check using a quality checker on the detected anti-patterns and posi-patterns using multimodal large language models (LLMs), to verify the accuracy and relevance of the detected patterns;
generating reports that verify anti-patterns and posi-patterns and provide recommendations to the user, wherein the reports include a video clip featuring the section where anti-pattern or posi-patterns occurred.

15. The system of claim 14 wherein the generated reports are displayed to the user on a user interface integrated within the online learning platform.

16. The system of claim 14, wherein the data collector also collects the data and time of the question asked during the online learning session and the quiz details, including the time taken to attempt the quiz and the correct and incorrect answers.

17. The system of claim 14 further comprises;
an API that provides a plurality of metrics, including the list of visited URLs and user details.

18. The system of claim 14 wherein the AI engine utilizes a Vision Large Language Model (LLM-V) capable of interpreting and understanding images paired with text, enabling the AI engine to process and analyze multimodal data.

19. The system of claim 14 wherein the analyzer utilizes computer vision techniques to analyze video recordings to determine the user's learning behavior for pattern detection.

20. The system of claim 14 wherein the learning pattern detector utilizes machine learning algorithms to automatically detect anti-patterns and posi-patterns based on video and data analytics.

21. The system of claim 14 wherein the generated reports, including video clips, are stored in a cloud database in JSON format.
